(19) United States
(12) Patent Application Publication
(10) Pub. No.: US 2023/0092893 A1
(43) Pub. Date: Mar. 23, 2023

(54) ULTRAVIOLET GERMICIDAL DOOR HANDLE

(71) Applicant: United States of America as represented by the Administrator of NASA, Washington, DC (US)

(72) Inventor: Jonathan A. Lee, Madison, AL (US)

(21) Appl. No.: 17/482,109

(22) Filed: Sep. 22, 2021

(51) Int. Cl. E05B 1/00; A61L 2/24; A61L 2/10

(52) U.S. Cl. CPC E05B 1/0069; A61L 2/24; A61L 2/10; A61L 2202/11; A61L 2202/14

(57) ABSTRACT

An ultraviolet germicidal door handle includes a germicidal ultraviolet light source mounted at an end of the door handle that includes a capacitive or other MEMS sensor(s) which detects a user's touch or movement operation and initiates activation and timing operation of the ultraviolet germicidal light source. Ultraviolet germicidal light emitted from the light source is directed as a column along a circumferential length of the door handle, thereby providing a germicidal action/effect on all touching surfaces of the door handle.

### ORIGIN OF THE INVENTION

The invention described herein was made by an employee of the United States Government and may be manufactured and used by or for the government of the United States of America for governmental purposes without the payment of any royalties thereon or therefor.

### FIELD OF THE INVENTION

This invention relates generally to door handles. More specifically, the invention is directed to germicidal door handles that employ ultraviolet light as a germicidal agent.

### BACKGROUND OF THE INVENTION

As is known, conventional door handles suffer from actual or perceived cleanliness and/or sanitation issues. Such issues arise because infectious viral and/or bacterial agents may be indirectly transmitted from person-to-person when an infected person touches a surface and leaves the infectious agents on that surface. An uninfected person subsequently touches the same surface and communication of the infectious agent to that subsequent person occurs. Examples of communicable diseases/infectious agents that may possibly be spread in this manner include—but are not limited to—chicken pox, conjunctivitis, Hepatitis A and B, herpes simplex, influenza, measles, mononucleosis, pertussis, adeno/rhino virus, and SARS-CoV-2.

### SUMMARY OF THE INVENTION

An advance in the art is made according to aspects of the present invention directed to an ultraviolet germicidal door handle that employs ultraviolet light as a germicidal agent.

In sharp contrast to the prior art, an ultraviolet germicidal door handle according to aspects of the present invention advantageously irradiates the door handle along its length and is attachable/detachable and optionally adaptable to a variety of cross-sectional door handle shapes.

In an illustrative embodiment, an ultraviolet germicidal door handle according to aspects of the present invention includes a germicidal ultraviolet light source mounted at an end of the door handle that includes a capacitive or other sensor which detects a user's touch and initiates activation and timing operation of the ultraviolet germicidal light source. Ultraviolet germicidal light emitted from the light source is directed as a column along a circumferential length of the door handle, thereby providing a germicidal action/effect on all touching surfaces of the door handle.

### BRIEF DESCRIPTION OF THE DRAWING

A more complete understanding of the present invention may be realized by reference to the accompanying drawing in which:

FIG. 1 shows a lifespan of SARS-CoV-2 virus in air and on a variety of surfaces;

FIG. 2 shows a schematic diagram illustrating classifications of ultraviolet light and its germicidal range/effectiveness according to aspects of the present invention;

FIG. 3 shows a schematic diagram of an illustrative ultraviolet germicidal door handle and attachable/detachable ultraviolet light device according to aspects of the present invention;

FIG. 4 shows a schematic diagram of an illustrative ultraviolet germicidal door handle and attachable/detachable ultraviolet light device in operation to provide germicidal action/effect according to aspects of the present invention;

FIG. 5 shows a flow diagram illustrating a generalized operating sequence of an ultraviolet germicidal door handle according to aspects of the present invention;

FIG. 6 shows a schematic cutaway-view of an illustrative ultraviolet germicidal door handle including an interior battery compartment and attachable/detachable light device according to aspects of the present invention; and

FIG. 7(A) shows a schematic diagram of an illustrative ultraviolet germicidal pull door handle and attachable/detachable ultraviolet light device according to aspects of the present invention;

FIG. 7(B) shows a schematic diagram of an illustrative ultraviolet germicidal pull door handle and attachable/detachable ultraviolet light device in operation to provide germicidal action/effect according to aspects of the present invention;

FIG. 8 shows a schematic cutaway-view of an illustrative ultraviolet germicidal pull door handle including an interior battery compartment and attachable/detachable ultraviolet light device according to aspects of the present invention;

FIG. 9 shows a schematic perspective view of an attachable/detachable ultraviolet light device and illustrative indicator(s), switch(es), and port(s) according to aspects of the present invention

FIG. 10 shows a schematic end view of an attachable/detachable/conformable ultraviolet light device having a conformable attachment clamp that permits attachment to door handles having irregular or not-round cross-sectional shape according to aspects of the present invention; and

FIG. 11 shows a schematic view of an attachable/detachable/conformable ultraviolet light device including a controller, internal battery and sensors according to aspects of the present invention.

The illustrative embodiments are described more fully by the Figures and more detailed description. Embodiments according to this disclosure may, however, be embodied in various forms and are not limited to specific or illustrative embodiments described in the drawing and detailed description.

### DESCRIPTION OF THE PREFERRED EMBODIMENT(S)

The following merely illustrates the principles of the invention. It will thus be appreciated that those skilled in the art will be able to devise various arrangements which, although not explicitly described or shown herein, embody the principles of the invention and are included within its spirit and scope.

Furthermore, all examples and conditional language recited herein are intended to be only for pedagogical purposes to aid the reader in understanding the principles of the invention and concepts contributed by the inventor(s) to furthering the art and are to be construed as being without limitation to such specifically recited examples and conditions.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Unless otherwise explicitly specified herein, the FIGs comprising the drawing are not drawn to scale.

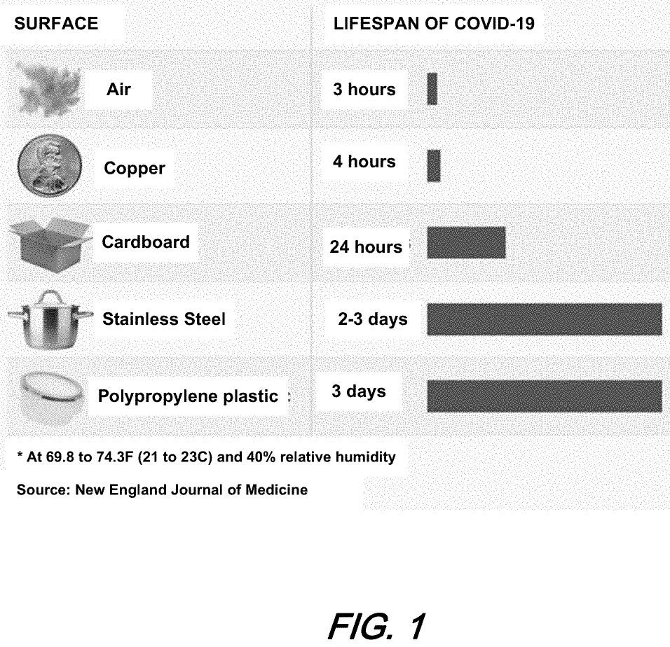

By way of some further background, it is noted that according to the Centers for Disease Control and Prevention (CDC), it is believed possible for people to be infected with SARS-CoV-2—the virus that causes COVID-19—through contact with contaminated surfaces or objects (fomites). To better understand such infection vectors, CDC has reported how long SARS-CoV-2 can survive on a variety of porous and non-porous surfaces. On porous surfaces, CDC reports an inability to detect viable virus within minutes to hours; on non-porous surfaces, viable virus can be detected for days to weeks. FIG. 1 shows a summary of the lifespan of SARS-CoV-2 virus in air and on a variety of surfaces.

Fortunately, from studies of surface cleaning focused on other microbes, it is known that a 90-99.9% reduction of microbe levels may be possible depending on the cleaning method and the surface being cleaned. To substantially inactivate SARS-CoV-2 on surfaces, the surface must be treated with a suitable disinfectant product or technology that has been shown to be effective against the virus.

One such known technology exhibiting an efficacy for inactivating SARS-CoV-2 virus (and others) is ultraviolet radiation (UV radiation) and in particular UV-C radiation—which is the highest energy portion of the UV radiation spectrum.

UV-C radiation is a known disinfectant for air, water, and nonporous surfaces. UV-C radiation has been used for decades to reduce the spread of bacteria, such as tuberculosis. For this reason, UV-C lamps are often called “germicidal” lamps.

UV-C radiation has been shown to destroy the outer protein coating of SARS-Coronavirus. This destruction ultimately leads to inactivation of the virus.

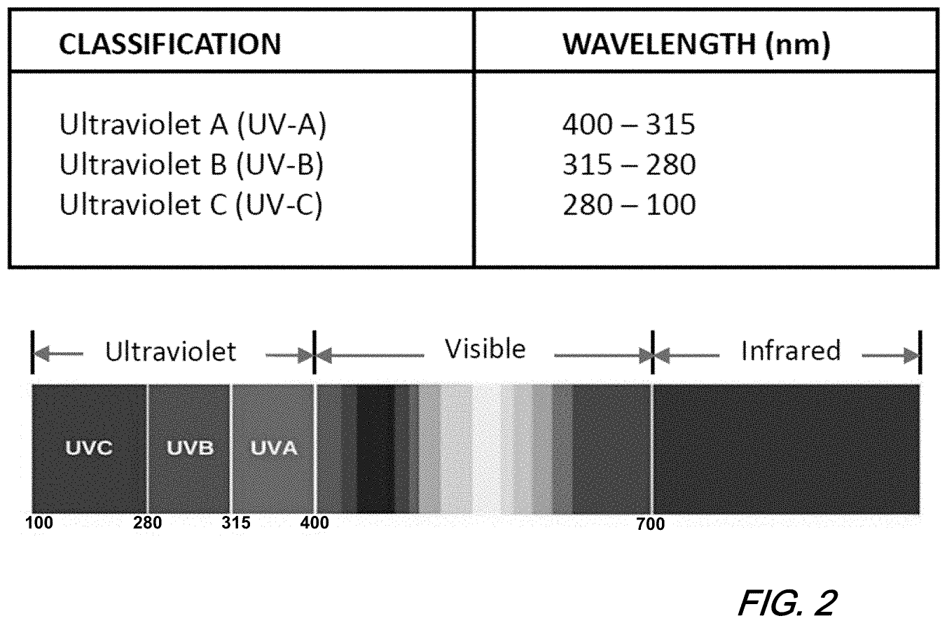

FIG. 2 shows a schematic diagram illustrating classifications of ultraviolet light and its germicidal range/effectiveness according to aspects of the present invention. While not specifically shown in the figure, light-emitting diodes (LEDs) that produce UV-C radiation are now available. Typically, LEDs emit a very narrow wavelength band of radiation. Contemporary LEDs have peak wavelengths at 265 nm, 273 nm, and 280 nm—among others—which may be advantageously employed in ultraviolet germicidal door handles according to aspects of the present invention.

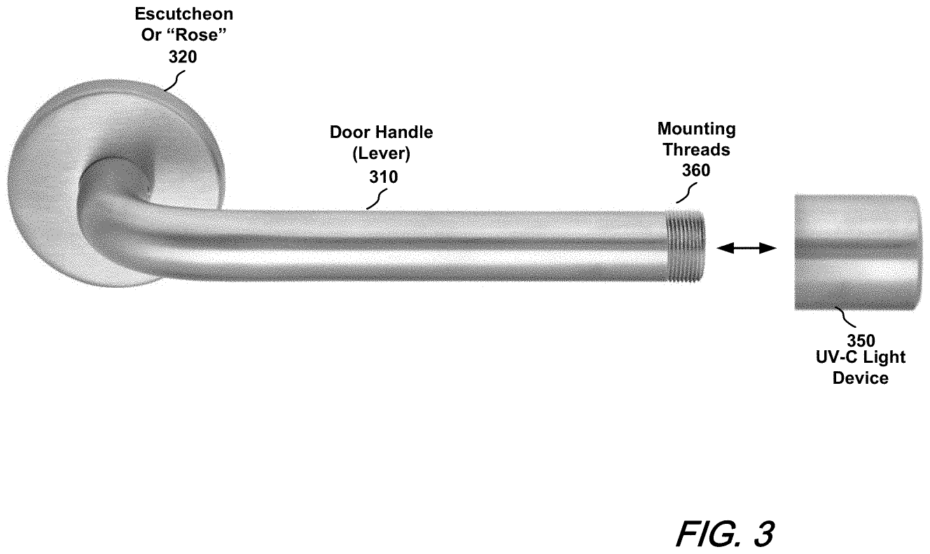

Turning now to FIG. 3, there it shows a schematic diagram of an illustrative ultraviolet germicidal door handle and attachable/detachable ultraviolet light device according to aspects of the present invention. Illustrated in that figure, a lever-type door handle 310 is shown along with a paired escutcheon plate or rose assembly 320. Generally, such lever-type handles are characterized by a rotational operation about an axis substantially perpendicular to the plane of a door to which they are mounted. This rotational operation of the door handle actuates a latch bolt and/or deadlatch to disengage from a strike plate or strike box and therefore allows the door to open.

Note that for the purposes of this disclosure and description of invention, the particular and specific details of latch mechanisms, strike plates, and doors are not necessary to understand the instant invention. As a result, such details are not described herein as those skilled in the art will readily understand and appreciate such structures and assemblies as well as their relationship(s) to the instant invention of germicidal ultraviolet door handles.

With continued reference to FIG. 3, also shown in the figure is ultraviolet light device 350 which is illustrated as threadably attachable/detachable to/from the door handle 310 through the effect of the mounting threads 360 formed on the distal end of the door handle. As will be understood and appreciated by those skilled in the art, the ultraviolet light device 350 will include internal mating threads—not specifically shown—such that it may be threaded on-to or off-from the door handle mounting threads 360 by rotating it relative to the door handle.

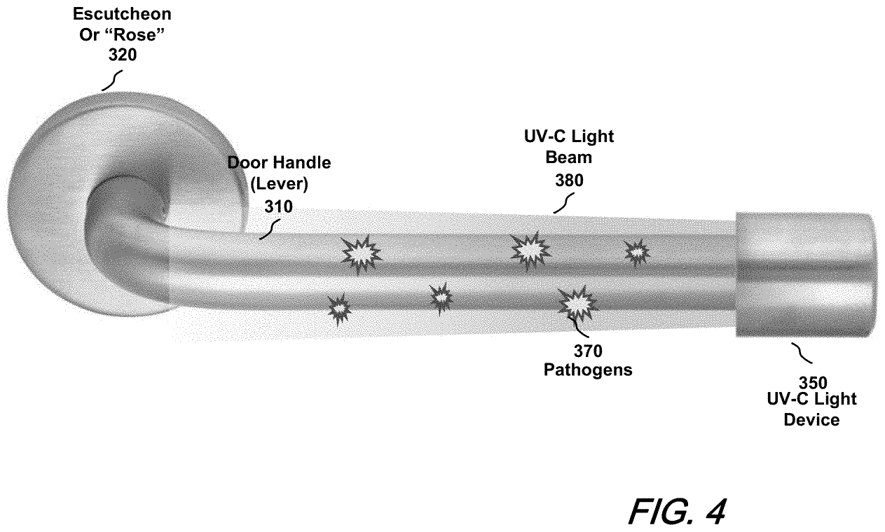

FIG. 4 shows a schematic diagram of an illustrative ultraviolet germicidal door handle and attachable/detachable ultraviolet light device in operation to provide germicidal action/effect according to aspects of the present invention. As shown in that figure, the door handle 310 has disposed on its surface certain pathogens 370 that may have resulted from a prior use of the door handle by an infected person or other transmission mechanism.

According to an aspect of the present disclosure, when such an “infectious” door handle is subsequently operated—for example, by a rotation of the handle—a germicidal ultraviolet light (UV-C) beam 380 is generated by the ultraviolet light device 350 and directed along the length of the door handle to effectively inactivate the pathogens 370.

As will be understood and appreciated by those skilled in the art, when the germicidal ultraviolet light beam is directed along the length of the door handle as now disclosed by the present invention, the entire circumference of the door handle along its entire operational touching surface may be effectively illuminated and therefore disinfected by its germicidal action of the UV-C light.

At this point it is noted that activating operation of the germicidal ultraviolet light may be initiated by any of a number of activities including user touch (i.e., capacitance) or movement (i.e., MEMS or other motion detectors, accelerometers, etc.) that may advantageously be included in the germicidal ultraviolet light device that is attached to the door handle.

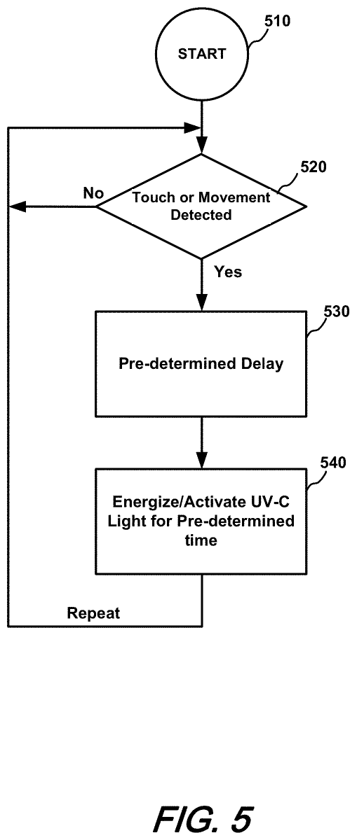

FIG. 5 shows a flow diagram illustrating a generalized operating sequence of an ultraviolet germicidal door handle according to aspects of the present invention. With reference to that figure, the flow diagram. With reference to that figure and flow diagram, operation is initiated at Block 510 and a continuous determination is made at Block 520 whether or not a user has touched the door handle or otherwise moved the handle. As previously noted, such touch may be determined by known techniques including capacitance sensor(s) located in the UV-C light device or alternatively, by a microelectromechanical system (MEMS) accelerator or switch that may likewise be located in the UV-C light device.

Upon determining that a touch or movement has occurred, a predetermined delay is performed at Block 530 which provides sufficient time for a person who operated the door handle to have entered/exited. Subsequently, the UV-C light device is activated for a predetermined time to disinfect the door handle. Subsequent to such disinfection, the monitoring/determining/disinfecting operation beginning at Block 520 is continuously performed.

As will be understood and appreciate by those skilled in the art, the user definable pre-determined delay and pre-determined energization of the UV-C disinfection may be altered depending upon the specific type of pathogen(s) for which the disinfection is performed. For example, a delay of 5—15 seconds—or more—may be required along with any sensing of additional or continuous door handle movement may be required prior to activation/energization of the UV-C light. Similarly, the light may be activated for a defined, pre-determined time—for example 15-30 seconds or more. Such delay and/or timing may be pre-programmed into the light device or changed via user programming from USB, WiFI or Low Power wireless communications between the light device and a computer and/or smartphone, for example.

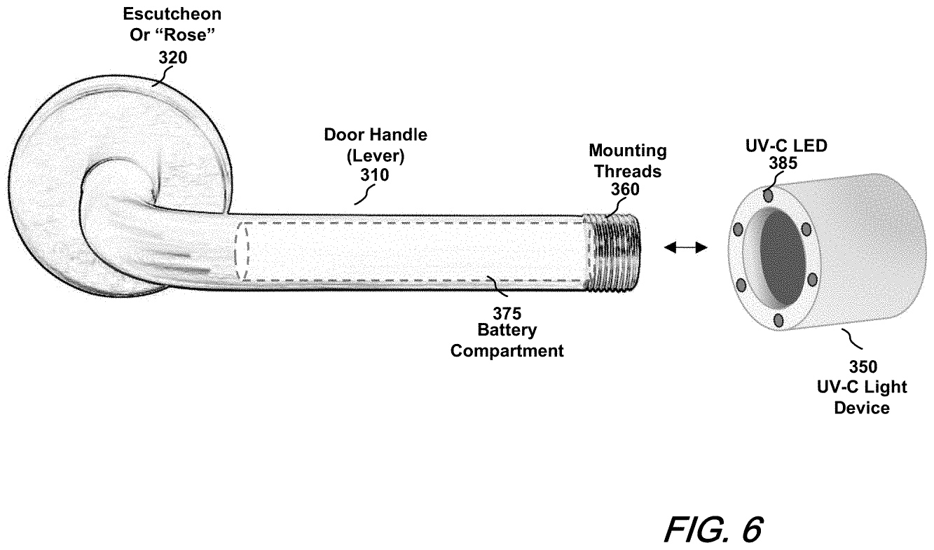

FIG. 6 shows a schematic cutaway-view of an illustrative ultraviolet germicidal door handle 310 including an interior battery compartment 375 and attachable/detachable ultraviolet light device 350 according to aspects of the present invention. As illustrated, a battery compartment 350 is located in an inner portion of the door lever. The battery compartment of such a configuration will contain batteries that power the operation of the UV-C light device. Electrical connections to the UV-C light device are made when the light device is attached to the handle. Note that while this figure shows the interior handle battery compartment and an illustrative threaded attachment/detachment mechanism, as we shall show and describe later in this disclosure the instant invention is not so limited as alternative attachment/detachment mechanisms and battery locations are possible.

Shown further in FIG. 6 are a plurality of UV-C light emitting diodes 380 arranged around a perimeter of an end of the light device 350 that generally faces the length of the door handle 310 when the light device 350 is attached to the handle 350. As will be readily appreciated by those skilled in the art, when the light device 350 is attached to the door handle 350, the perimeter arranged, handle facing LEDs 385 may illuminate the entire length and perimeter of the door handle 310 with disinfecting UV-C light when the LEDs are energized. As noted, the energizing of the LEDs is initiated by a touch of a person opening the door, or alternatively, when a sensor within the light device detects such opening by other sensory action such as a MEMS accelerator.

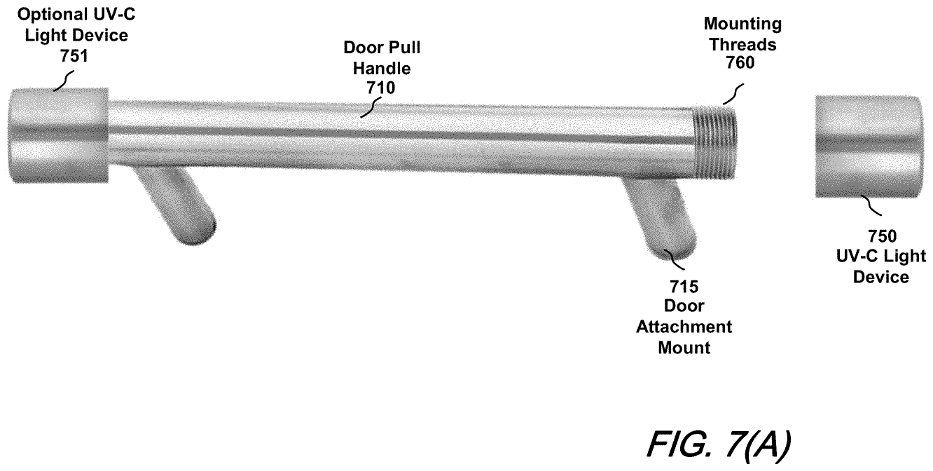

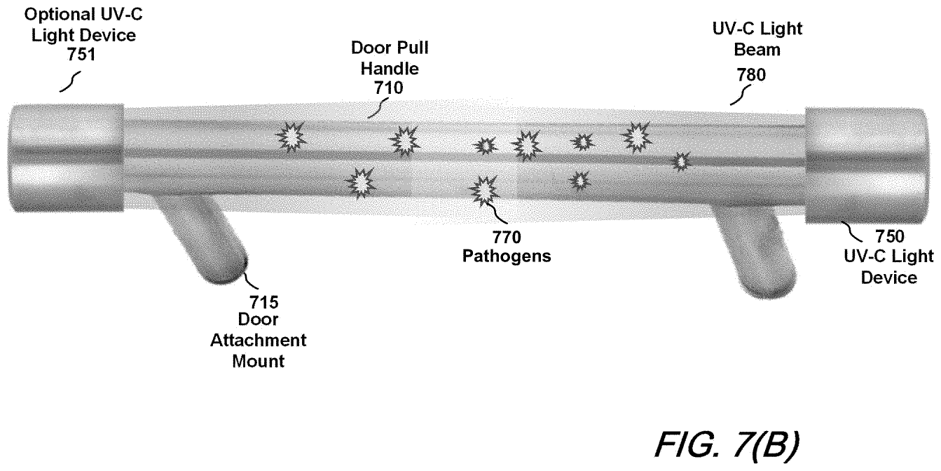

FIG. 7(A) shows a schematic diagram of an illustrative ultraviolet germicidal pull door handle and attachable/detachable ultraviolet light device according to aspects of the present invention. Similar to the door lever configuration shown previously, the pull door handle 710 and attachable/detachable ultraviolet light device 750 are illustrated as threadably attachable/detachable to/from the pull door handle 710 through the effect of the mounting threads 760 formed on one or more ends of the pull door handle. As will be understood and appreciated by those skilled in the art, the ultraviolet light device 750 will include internal mating threads—not specifically shown—such that it may be threaded on-to or off-from the door handle mounting threads 760 by rotating it relative to the door handle.

Of particular advantage to the illustrative configuration shown in FIG. 7(A), it is configurable such that more than one attachable/detachable ultraviolet light device 750 may be connected to the single pull door handle. Such configuration may provide additional germicidal UV-C light, from both light devices 750 and 751 shown illustratively located at a top and bottom of the pull door handle 710.

FIG. 7(B) shows a schematic diagram of an illustrative ultraviolet germicidal pull door handle and attachable/detachable ultraviolet light device in operation to provide germicidal action/effect according to aspects of the present invention. As shown in that figure, the pull door handle 710 has disposed on its surface certain pathogens 770 that may have resulted from a prior use of the door handle by an infected person or other transmission mechanism.

According to an aspect of the present disclosure, when such an “infectious” door handle is subsequently operated—for example, by a person's touch or a sensed opening of the door—a germicidal ultraviolet light (UV-C) beam 780 is generated by the ultraviolet light device 750 and directed along the length of the door handle to effectively inactivate the pathogens 770. As noted previously, both ends of the pull door handle 710 may be configured with an ultraviolet light device 750 and 751 such that an enhanced germicidal effect is produces as UV-C light beam(s) are generated at both ends of the pull door handle and directed in opposite directions along its length and full circumference.

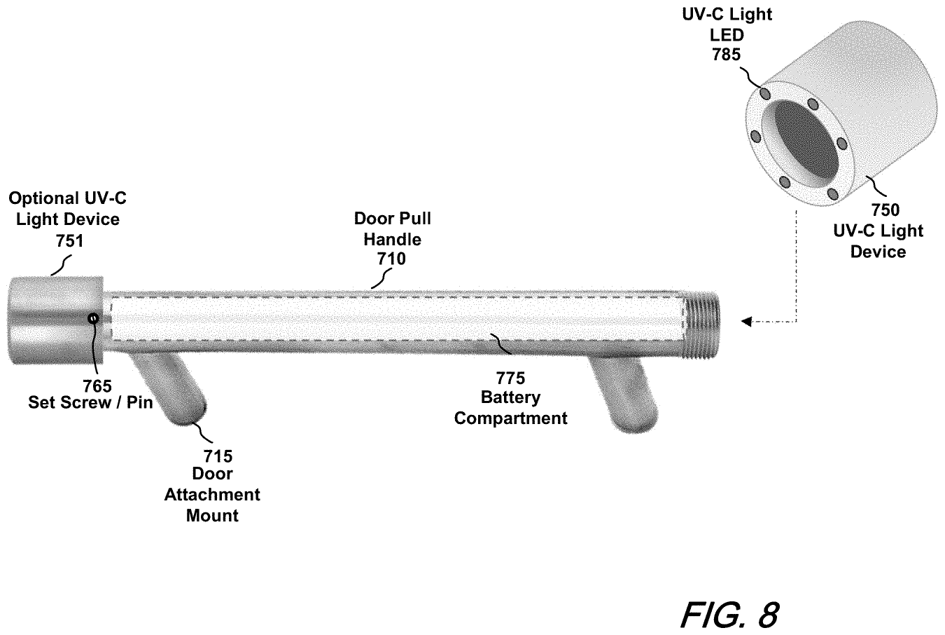

FIG. 8 shows a schematic cutaway-view of an illustrative ultraviolet germicidal pull door handle 710 including an interior battery compartment 770 and attachable/detachable ultraviolet light device 750 according to aspects of the present invention. As illustrated, the battery compartment 775 is located in an inner portion of the pull door handle 710 and will contain batteries during operation (not specifically shown). Electrical connections to the UV-C light device (not specifically shown) are made when the light device 750 is attached to the handle 710 which, in this illustrative configuration is achieved by mated mounting threads 760 formed on the handle 710. Note that while this figure shows an interior handle battery compartment 775 and an illustrative threaded attachment/detachment mechanism 760, as we shall show and describe later in this disclosure, the instant invention is not so limited as alternative attachment/detachment mechanisms and battery locations are possible.

FIG. 8 additionally shows the light device 750 in perspective such that a plurality of UV-C LED lights disposed around a perimeter of the device. As will be readily understood and appreciated, the arrangement and number of LED lights will be determined by the shape/length etc., of the particular type of door handle to which the light device is to be attached.

Shown further in FIG. 8 is an alternative attachment mechanism for the light device 750 that is illustratively attached to a lower end of the pull type door handle 710. As illustrated, the lower light device is attached/fixed to the door handle through the effect of a set screw 765 or attachment pin that engages both the light device and the handle such that they are secured together. When so configured, removal of the light device may be achieved by loosening the set screw 765 or removal of the attachment pin.

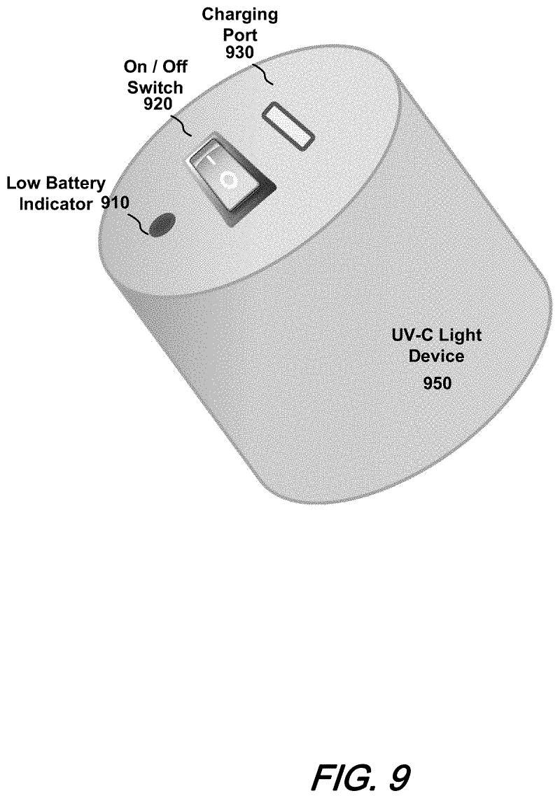

FIG. 9 shows a schematic perspective view of an attachable/detachable ultraviolet light device 950 and illustrative indicator(s), switch(es), and port(s) according to aspects of the present invention. With reference to that figure, shown is an illustrative light device 950 having a low battery indicator light/lamp/LED 910 that may be activated when battery power drops below a certain, pre-determined threshold. Shown further is a charging port 930, that is illustratively indicated as a universal serial bus (USB) type port although other types—including inductive-type charging may be employed. Finally, an on/off—I/O switch 920 is shown that may also include an LED indicative that the device is on and operational.

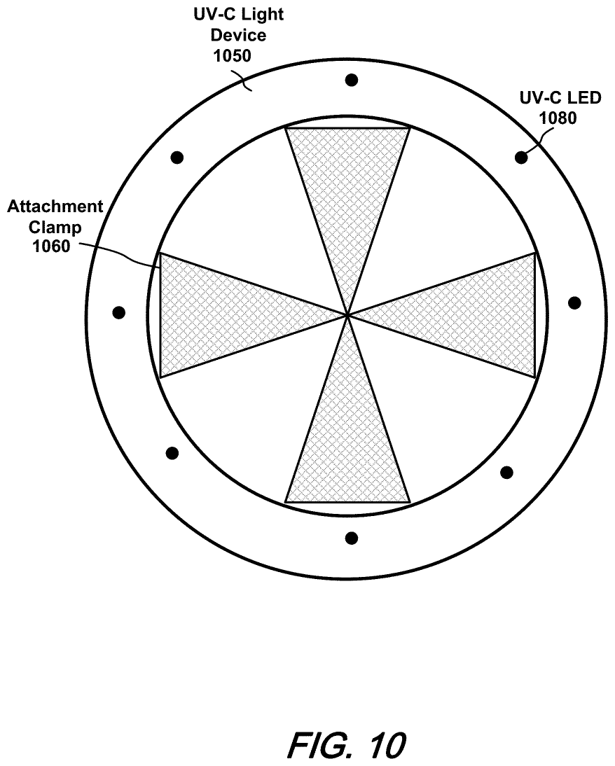

FIG. 10 shows a schematic end view of an attachable/detachable/conformable ultraviolet light device 1050 including a plurality of UV-C LED lights 1080 disposed along a perimeter face according to aspects of the present invention. Shown in that figure is an attachment clamp 1060, which may be threaded into or securely attached to the light device. As those skilled in the art will understand and appreciate, such an attachment clamp may be constructed from a conductive material (metal, or metalized plastic) that conforms when the light device is placed upon and urged onto an end of the door handle. Such clamps may be constructed from materials that mechanically and electrically contact the door handle and provide a “push fit” engagement. These types of clamps/fittings may provide a semi-permanent or removable attachment as application needs dictate. Of further advantage, with an appropriate attachment clamp 1060 affixed to the light device 1050, the light device 1050 may be attached to door handles having a cross-sectional shape that is not round. For example the door handle may be flat or irregularly shaped in cross-section. This is achieved as the attachment clamp 1060 is conformable to the cross-sectional shape of the door handle onto which it is push-fit.

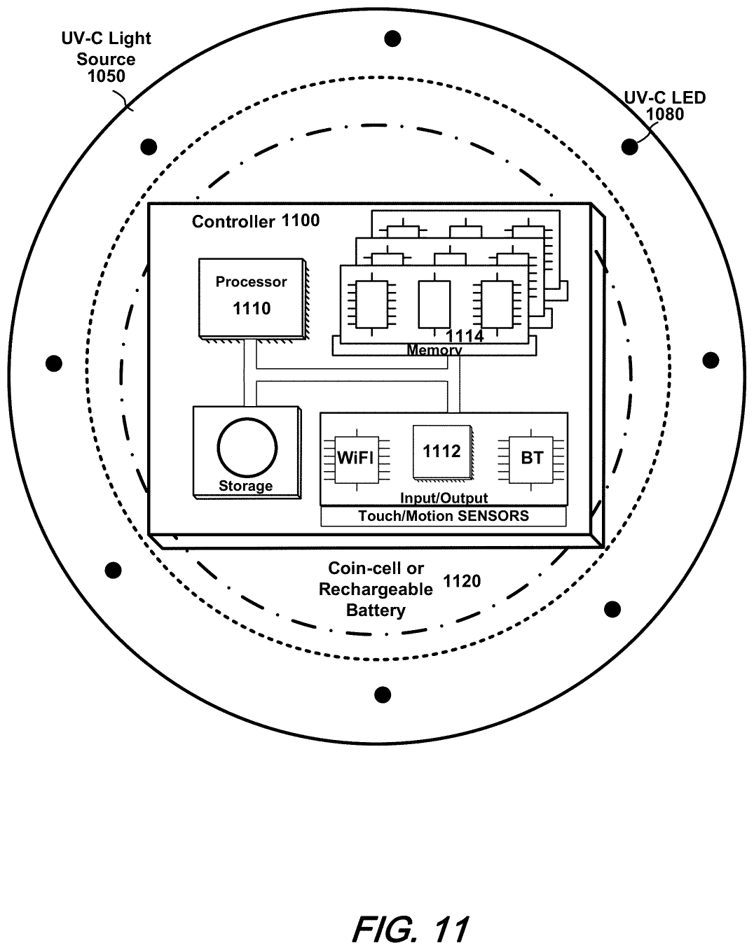

FIG. 11 shows a schematic view of an attachable/detachable/conformable ultraviolet light device having a controller 1100 and internal battery 1120 according to aspects of the present invention. As shown, the light device controller may include any indicators, switches, and port(s) described previously, along with programmable logic 1110 and internal battery power 1120. As will be understood and appreciated by those skilled in the art, the programmable logic may include additional input/output 1112 and memory 1114 that advantageously provides for convenient user programmability, recharging, or control via USB or other technologies—i.e., wireless including low power wireless (BlueTooth) and WiFi. When so configured, each individual ultraviolet light device according to the present invention may be monitored/controlled from a central location or other devices including contemporary cellular telephones.

At this point, while we have presented this disclosure using some specific examples, those skilled in the art will recognize that our teachings are not so limited. Accordingly, this disclosure should be only limited by the scope of the claims attached hereto.

1. An ultraviolet germicidal door handle comprising:
the door handle;
a mounting plate for mounting the door handle to a door; and
an attachable/detachable light device that is attached to an end of the door handle, the light device configured to generate ultraviolet (UV-C) light and direct the generated light along an entire touchable length and circumference of the door handle such that pathogens on the door handle are deactivated.

2. The ultraviolet germicidal door handle according to claim 1 wherein the light device includes a plurality of UV-C light emitting diodes (LEDs) disposed along a perimeter of a face of the light device that faces the length of the door handle.

3. The ultraviolet germicidal door handle according to claim 2 further comprising mounting threads formed in the end of the door handle to which the light device is threadably attached.

4. The ultraviolet germicidal door handle according to claim 2 further wherein the light device is push-fit attached to an end of the door handle through the effect of an attachment clamp attached to the light device.

5. The ultraviolet germicidal door handle according to claim 2 wherein the light device includes one or more sensors for detecting touch and/or movement of the door handle, said sensors selected from the group consisting of capacitance sensors, microelectromechanical system (MEMS) accelerometers, and MEMS switches.

6. The ultraviolet germicidal door handle according to claim 5 wherein the door handle is a type selected from the group consisting of rotating handles and non-rotating pull handles.

7. The ultraviolet germicidal door handle according to claim 5 wherein the door handle is a non-rotating pull type door handle and includes two light devices, one located at one end of the door handle and the other located at another end of the door handle, each of the two light devices configured to generate UV-C light and respectively direct the generated light along the length and circumference of the door handle in opposite directions.

8. The ultraviolet germicidal door handle according to claim 6 wherein the door handle includes a battery compartment within an interior portion of the touchable length.

9. The ultraviolet germicidal door handle according to claim 2 wherein the light device is attached to the door handle by a set screw.

10. The ultraviolet germicidal door handle according to claim 4 wherein the door handle exhibits a non-circular cross-sectional shape and the light device is push-fit attached to the end of the door handle.

11. The ultraviolet germicidal door handle according to claim 2 wherein the light device includes a programmable controller, a battery power source, and one or more sensors for detecting a user touch and/or door handle operation.

12. The ultraviolet germicidal door handle according to claim 11 wherein the programmable controller is configured to delay activation of the UV-C light for a pre-determined period after a sensed touch or movement and subsequently activate the UV-C light for a pre-determined period after the delay period.

13. The ultraviolet germicidal door handle of claim 2 wherein the light device includes a low battery indicator, an on/off indicator, and a charging port.

14. The ultraviolet germicidal door handle of claim 11 wherein the light device includes one or more wireless communications systems and protocols selected from the group consisting of wireless fidelity (WiFI) and short-range wireless (BlueTooth) and is configured to be user programmable/monitorable via these communications systems and protocols.

15. A method of operating an ultraviolet germicidal door handle including an attachable/detachable light device attached to an end of the door handle, the light device configured to generate ultraviolet (UV-C) light and direct the generated light along an entire touchable length and circumference of the door handle such that pathogens on the door handle are deactivated, the method comprising:
detecting a user touching the door handle or the door handle moved by a user;
delaying for a pre-determined period of time;
energizing/activating the light device for a pre-determined period of time; and
continuously repeating the detecting, delaying, and energizing/activating.

16. The method of claim 15 wherein the light device includes programmable logic and communications interface(s), the method further comprising:
updating the pre-determined periods via the communications interfaces.

17. The method of claim 16 further comprising monitoring an operational status of the ultraviolet germicidal door handle via the communications interfaces.
